(19) United States
(12) Patent Application Publication
(10) Pub. No.: US 2010/0136629 A1
(43) Pub. Date: Jun. 3, 2010

(54) ENHANCED ETHANOL FERMENTATION USING BIODIGESTATE

(76) Inventors: Xiaomei LI, Edmonton (CA); Tiejun GAO, Edmonton (CA)

Correspondence Address: MCCARTER & ENGLISH, LLP BOSTON, 265 Franklin Street, Boston, MA, 02110, US

(21) Appl. No.: 12/611,162

(22) Filed: Nov. 3, 2009

Related U.S. Application Data
(60) Provisional application No. 61/198,224, filed on Nov. 4, 2008.

(51) Int. Cl. C12P 19/00; C12P 7/06

(52) U.S. Cl. 435/72; 435/161

(57) ABSTRACT

Methods and systems for enhancing ethanol production using a suspending fluid are described. The suspending fluid includes organic material that has at least partially been anaerobically digested and anaerobic microorganisms, and is substantially free of non-anaerobic microorganisms. Also described are methods and systems for hydrolyzing a feedstock for fermentation that include hydrolyzing a feedstock suspension. The feedstock suspension can include feedstock that includes complex sugars, and a suspending fluid, wherein the suspending fluid includes organic material that has at least partially been anaerobically digested and anaerobic microorganisms, and is substantially free of non-anaerobic microorganisms.

### REFERENCE TO RELATED APPLICATIONS

This application claims the benefit of the filing date under 35 U.S.C. §119(e) to U.S. Provisional Application No. 61/198,224, filed on Nov. 4, 2008, the entire contents of which, including the specification and drawings, are incorporated herein by reference.

### BACKGROUND OF THE INVENTION

Ethanol has many commercial uses, and, for example, can be used for combustion as a fuel or a fuel additive. Ethanol (also known as bioethanol) can be produced by fermenting sugars contained in a feedstock. The fermentation can be carried out by microorganisms, such as yeasts or bacteria, that can convert the sugars into ethanol through biochemical processes. The feedstock can include organic material, generally plant material, that contains sugars. Examples of plant material that can be used as feedstock include plants that produce and store simple sugars (e.g., sugar cane and sugar beets), plants that produce and store starch (e.g., grains, such as corn and wheat), and other plant material rich in cellulose and/or hemi-cellulose (e.g., agricultural or forestry residues, such as plant stalks and leaves).

The production of ethanol by fermentation can require many materials in addition to feedstock and microorganisms. These materials can include fresh process water, which can be added to the feedstock to create a suspension of feedstock for the microorganisms to ferment, and nutrient supplements, especially nitrogen supplements (e.g., urea or ammonium compounds), which can provide the necessary nutrients to the microorganisms performing the fermentation. However, these materials can be expensive, and can prohibitively increase the costs of ethanol production, which is one of the major obstacles that presents the ethanol-based fuel from competing economically with gasoline. For example, water consumption in a conventional ethanol plant is about 10 gPM per million gallons annual ethanol production. This means a huge amount of fresh water will be consumed for massive bioethanol production in the near future. However, no much research effort and related actions have been put forward to date to alleviate the problem.

Feedstock for ethanol fermentation can include complex sugars, such as polysaccharides, which generally are difficult for microorganisms to ferment into ethanol. To assist in fermentation of complex sugars contained therein, the feedstock can be subjected to hydrolysis reactions, where the complex sugars are converted to simpler sugars that can more readily be converted by microorganisms into ethanol. The hydrolysis process can also be expensive, in part because of the need for materials such as fresh water and enzymes that perform the conversion.

In addition, traditional ethanol plants have also been rightly criticized for their lack of energy efficiency. The biggest loss in energy efficiency generally comes from use of fossil fuels for distillation and drying of distillers grains—the wet residues in the fermentation beer after the produced ethanol is distilled.

Organic waste, such as municipal wastewater or livestock manure, can release greenhouse gases, such as methane and carbon dioxide, and can be a source of air, soil, and water pollution. Anaerobic bio-digesters can process the organic waste by treatment with organisms, which can be obligate or facultative bacteria and/or archaea. These organisms can, using biochemical reactions, convert organic material into a variety of products. Among these products are a mixture of gases, generally referred to as biogas, and a mixture of liquids and solids, generally referred to as biodigestate. Biodigestate is generally treated as a waste material.

### SUMMARY OF THE INVENTION

The present invention provides methods and systems for enhancing ethanol production and deriving value-added products from biodigestate, which is traditionally considered waste material. The methods and systems of the invention are partly based on the discovery that biodigestate and different fractions thereof do not inhibit the activities of many enzymes required for microorganism-based fermentation process for ethanol production, and thus, can be used directly, without the addition of any fresh water or nutrient supplement, as the suspension fluid for the fermentation process. This not only provides a useful utilization of biodigestate—traditionally considered a waste material—but also saved valuable resources, such as fresh water and nutrient supplement. The methods and systems of the invention are also partly based on the surprising discovery that biodigestate or certain fractions thereof provide enhanced ethanol yield compared to fresh water, thereby further increasing the cost efficiency of ethanol production using microorganism fermentation. While not wishing to be bound by any particular theory, it is possible that the observed enhanced ethanol production results from the presence of certain nutrients and other organic substances lacking in fresh water (such as water insoluble substances (WIS) and nutrients in the AD effluent), which nutrients and other organic substances may aid the final yield of ethanol fermentation. It is also possible that the observed enhanced ethanol production results from the presence of certain microorganisms in the anaerobic digestate that can synergize saccharification and fermentation of grain bioethanol production.

Combining the AD technology with the bioethanol production process not only enables turning anaerobic digest effluent into value-added products, but also assists bioethanol industry to achieve positive balance on energy consumption, bioethanol production, waste management, and environmental preservation in order to maximize its profit.

Thus one aspect of the invention provides a method for producing ethanol, comprising: (1) adding a suspending fluid to a feedstock to produce a fermentation suspension, wherein the suspending fluid comprises an organic material that has at least partially been anaerobically digested; (2) adjusting the pH of the fermentation suspension, if necessary, to a value conductive for fermentation; and (3) fermenting the fermentation suspension to produce ethanol, wherein the suspending fluid is substantially free of fresh water (e.g., exogenously added) or nutrient supplement.

In certain embodiments, the method further comprises inoculating the fermentation suspension with a microorganism capable of fermenting the fermentation suspension to produce ethanol. For example, the microorganism may be a yeast or a bacteria, or any other microorganism that can perform fermentation to produce ethanol. Exemplary ethanol-producing microorganisms include yeast Saccharomyces and bacteria Zymomonas, facultative anaerobic thermophilic bacteria strains such as those described in WO/88/09379, and genetically engineered microorganisms which otherwise would not produce significant ethanol with genetic engineering. See, for example, engineered E. coli with ADH and PDC enzymes from Zymomonas mobilis, Ingram et al., “Genetic Engineering of Ethanol Production in Escherichia coli.” Appl. Environ Microbiol. 53: 2420-2425, 1987; genetically modified photosynthetic Cyanobacteria, such as those described in U.S. Pat. No. 6,699,696; engineered Klebsiella oxytoca; and generally, see Dien et al., Bacteria engineered for fuel ethanol production: current status. Applied microbiology and biotechnology, 63: pages 258-266, 2003 (all incorporated by reference).

Preferred ethanol fermentation microorganisms can tolerate high concentration of ethanol (e.g., 10%, 15%, 20%, 25%, or 30%) in an AD-based fermentation broth. Preferred ethanol fermentation microorganisms can also breakdown non-starch cellulosic biomass efficiently, which can hydrolyze different non-grain biomass and convert it to single sugar molecule for fermentation. Recombinant DNA technology may be used to genetically enhance the traits of such fermentation microorganisms beneficial for ethanol fermentation.

In certain embodiments, the suspending fluid comprises, consists essentially of, or consists anaerobic biodigestate or effluents thereof. The anaerobic biodigestate may result from anaerobic digestion of an organic material (including any organic waste materials), such as an organic material comprising animal offal, livestock manure, food processing waste, municipal waste water, thin stillage, distiller's grains, and/or other organic materials.

In certain embodiments, the suspending fluid comprises, consists essentially of, or consists biodigestate as a whole. In other embodiments, the suspending fluid comprises, consists essentially of, or consists a fractioned anaerobic biodigestate. The fractioned anaerobic biodigestate may be a liquid fraction generated by removing substantially all solids from the anaerobic biodigestate by, for example, centrifugation. In certain embodiments, the supernatant of the centrifugation process performs the best in ethanol fermentation when there is certain level of suspended solids in the supernatant. Thus in certain embodiments, the supernatant is generated by centrifuging the AD effluent at 200 g, 400 g, 600 g, 800 g, 1000 g, 1500 g, 2000 g, 2500 g, 3000 g, 3500 g, 4000 g, 5000 g, 6000 g, 7500 g, or 10,000 g.

Alternatively, the liquid fraction may be generated by passing the anaerobic biodigestate through a screw press (such as a “FAN” brand screw press) or other similar devices.

Preferably, the AD digestate comes from a “healthy” batch of anaerobic digestion, in that the production of biogas in said healthy batch is optimum (vs. declining to near zero).

In certain embodiments, an amount of urea is added to the AD effluent to enhance yield. The AD may be used fresh, or may be stored for a period of time, such as 12 hrs, 1, 2, 3, 5, 7, 10, 2 weeks, 1 month, etc.

In certain embodiments, the liquid fraction contains about 1, 2,3, 4, 5, 6, 7, 8, 9, or 10% (preferably 3-9%) solids.

In certain embodiments, the liquid fraction may be further fortified by a nutrient recovered from the anaerobic biodigestate.

In certain embodiments, the fractioned anaerobic biodigestate is an ultrafiltration concentrate or an ultrafiltration permeate generated from a liquid fraction of the anaerobic biodigestate, wherein said liquid fraction is generated by removing substantially all solids from the anaerobic biodigestate.

In certain embodiments, the pH of the fermentation suspension is adjusted to below 6.0, (for example, between 4.0 and 5.0) for the best enzymatic catalysis.

In certain embodiments, the method further comprises distilling the post fermentation beer to collect ethanol without pre-removal of solids from the beer.

In certain embodiments, the feedstock is high-starch wheat, corn, or other high-starch crops.

In certain embodiments, the high-starch wheat, corn, or other high-starch crops is converted in the suspending fluid at least partially into simple sugars.

In certain embodiments, the conversion comprises (with no particular order and no limitation on repeats) mechanical grinding, heating with steam, reacting with an acid, liquefaction by using alpha-amylase, and/or saccharification by using glucoamylase.

In certain embodiments, pH is controlled in an optimal range required for the wheat or crop conversion reactions.

In certain embodiments, about 75% of the suspension fluid is added before liquefaction, and about 25% of the suspension fluid is added post liquefaction and before saccharification.

In certain embodiments, the amount of the high-starch wheat, corn, or other crop is up to about 28% (w/v), or up to 36% (w/v) in the suspension fluid.

In certain embodiments, the method further comprises adding cellulase, xylanase, and/or acid proteolytic enzyme to the suspension fluid.

In certain embodiments, the method further comprises incubation the fermentation mixture at about 30-50° C. (inclusive) for about 24 hours, 36, 48, or 72 hours.

In certain embodiments, the wet distillers grains resulting from ethanol distillation is fed to a livestock animal (e.g., swine, poultry, cattle, or fish) as feed, optionally with fortified neutrient elements, or used as fertilizers with enhanced nutrient value (e.g., nitrogen increment).

In certain embodiments, the suspending fluid is substantially free of non-anaerobic microorganisms.

In certain embodiments, the pH of the suspending fluid is adjusted to a value substantially incompatible for growth of non-anaerobic microorganisms.

In certain embodiments, the pH of the suspending fluid is adjusted to a value for optimal growth of fermentation microorganisms.

In certain embodiments, the nutrient supplement is a nitrogen supplement.

In certain embodiments, ethanol yield is enhanced or increased compared to an otherwise identical process using fresh water instead of the suspending fluid. Preferrably, ethanol production is increased by 5-15%, or 7-10%, when about 20-36% or 22-28% of wheat is used.

Another aspect of the invention provides a method for hydrolyzing a feedstock, wherein the feedstock comprises polysaccharides and wherein the hydrolyzed feedstock yields more ethanol when fermented than prior to hydrolysis, the method comprising: (1) adding a suspending fluid to the feedstock to produce a feedstock suspension, wherein the suspending fluid comprises organic material that has at least partially been anaerobically digested; and, (2) hydrolyzing the feedstock suspension such that at least a portion of the polysaccharides are converted into simple sugars, wherein the suspending fluid is substantially free of (exogenously added) fresh water or nutrient supplement.

In certain embodiments, the hydrolyzing step comprises (with no particular order and no limitation on repeats) mechanical grinding, heating with steam, reacting with an acid, liquefaction by using alpha-amylase, and/or saccharification by using glucoamylase.

It is contemplated that all embodiments of the invention described herein can be combined with any other embodiments, including those described under different aspects of the invention, unless explicitly disclaimed or obviously improper or non-applicable.

### BRIEF DESCRIPTION OF THE DRAWINGS

The above and other advantages of the present invention will become more apparent upon consideration of the following detailed description, taken in conjunction with the accompanying drawings, in which like reference characters refer to like parts throughout, and in which:

FIG. 1 is a flow chart 100 illustrating an exemplary process including steps 102, 104, and 106, for enhancing ethanol production in accordance with an embodiment of the present invention.

FIG. 2 illustrates a schematic view of an exemplary system 200 for enhancing ethanol production in accordance with an embodiment of the present invention. The system 200 may include a bio-digester 202, wherein organic waste material 204 is subject to anaerobic biodigestion to produce biodigestate and biogas. At least a part of the biodigestate 206 is transported to hydrolysis unit 214 for mixing with feedstock to produce a suspension. The hydrolysis may be done with enzyme 208 and/or acid 210 and/or heat 212 (e.g., in the form of steam, etc.). The resulting hydrolyzed feedstock suspension 218 is then fermented to produce ethanol 224. Alternatively, at least part of the biodigestate 216 can be transported to fermentator 220 directly and mixed with feedstock 218. Feedstock 222 can also be added to produce ethanol.

FIG. 3 is a flow chart 300 illustrating an exemplary process comprising steps 302, 304, and 306, for hydrolyzing a feedstock in accordance with an embodiment of the present invention.

FIG. 4 shows change of the specific gravity and potential ethanol content (% vol) from different fermentation groups up to 14 days at 22° C. Legend: groups: Tap H₂O: tap water, UF-per: Ultra Filtration (UF) permeate, UF-con: Ultra Filtration (UF) concentrate, S: granulate Sugar, SY: super Tubor yeast. Specific gravity (S.G.) was measured at fermenting day of 0, 4, 7, 11 and 14. Potential ethanol content was calculated based on Oechsle scale.

FIG. 5 is a comparison of wheat conversion in anaerobic digestate (AD) and tape-water by two-step enzymatic catalysis based on the glucose content (gram/gram of dry wheat).

FIG. 6 shows glucose yield after two-step enzymatic conversion with different contents of wheat in FAN-separated AD and in water.

FIG. 7 shows two procedures used in wheat conversion.

FIG. 8 shows ethanol yield in Simultaneous Saccharification and Fermentation (SSF) with AD and water with/without BG.

FIG. 9 shows dose-dependent ethanol yield in SSF of FAN-Separated anaerobic digestate (FSD) with different amounts of dry wheat.

FIG. 10 shows ethanol yield in SSF using two-step addition procedure of AD or H₂O. Legend: ¼ volume of either H₂O or FSD was added and incubated at 55° C. for additional 30 minutes before the G-ZYME® 480 (improved pre-saccharification and saccharification enzyme blend from GENENCOR®, Rochester, N.Y.) and OPTIMASH™ BG (beta glucanase/xylanase complex from GENENCOR®, Rochester, N.Y.) were added. W36 or W28: wheat 36 or 28 grams in 130 or 100 ml FSD or H₂O. H₂O, W28 as control. n=4 in each group.

FIG. 11 shows total solid (TS) and volatile solid (VS) in post-fermenting samples.

FIG. 12 is total nitrogen in post-fermenting solid from different groups.

FIG. 13 shows glucose yield from FSD catalyzed with OPTIMASH XL and Accellerase.

FIG. 14 shows Ethanol yield from SSF with OPTIMASH™ XL (high concentration cellulase/xylanase complex from GENENCOR®, Rochester, N.Y.) and Accellerase. *: Statistical significance.

FIG. 15 shows ethanol yield in FSD and H₂O-wheat mixture with/without FERMGEN™ (low pH protease from GENENCOR®, Rochester, N.Y.).

FIG. 16 shows ethanol yield in FSD/wheat and H₂O mixture with identical weights post fermentation. *: Statistical significance.

FIG. 17 shows nutrient value in wet distiller's grain (WDG) in fermentation using anaerobic digestate. “AD alone” represents the nutrient values of the anaerobic digestate alone before fermentation; “AD/wo centrif ” represents the nutrient values for whole AD (without centrifugation) fermented with wheat; “ADS, nnn rpm” represents the nutrient values for centrifuged AD at varied speeds (at “nnn rpm” respectively) fermented with wheat; “H₂O control” represents the nutrient values for wheat fermented in water; and “dry wheat” represents the nutrient values for grounded whole wheat without fermentation. “P-F” stands for “post fermentation.” For each group of bars, from left to right, are the values for crud protein, crud fiber, fat, and ash.

FIGS. 18 and 19 show the result of analyzing the various nutrient elements required in animal feeds as they are present in the various mash or WDGs. For each group of bars in FIGS. 18 and 19, from left to right, are the values for H₂O control, ADS (1000 rpm), ADS (4000 rpm), ADS (6000 rpm), AD alone, dry wheat, and AD/wo centrif, respectively.

FIG. 20 shows the calculated animal feed values for the various ADS (AD supernatant) batches as compared to fresh water alone. “TD” stands for “total digestible nutrients”; “NF” stands for “non fiber carbohydrate”; “DE” is “digestible energy”; “GE” is “gross energy”; and “ME” is “metabolizable energy.” For each group of bars, from left to right, are the values for H₂O control, ADS (1000 rpm), ADS (4000 rpm), ADS (6000 rpm), AD alone, dry wheat, and AD/wo centrif, respectively.

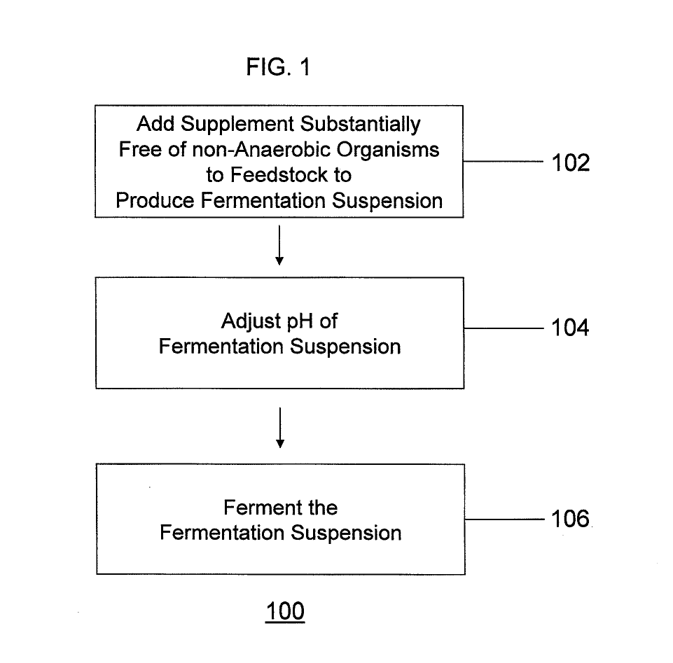

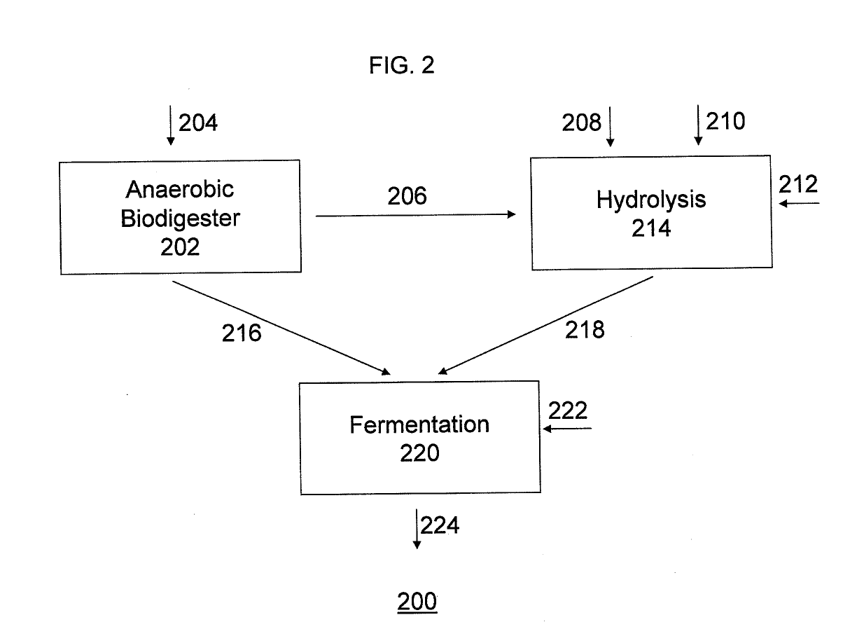

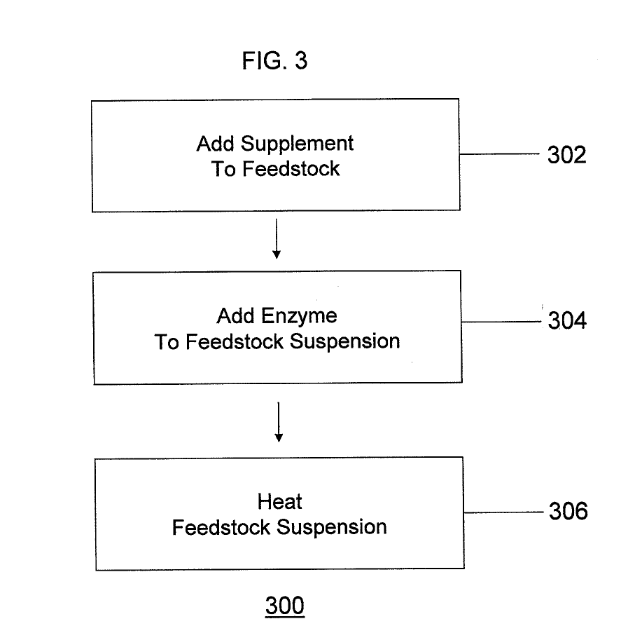

### DETAILED DESCRIPTION OF THE INVENTION

As noted hereinabove, it may be desirable to reduce or eliminate the use of fresh process water and/or of nutrient supplements (especially nitrogen supplements) during the fermentation process. Thus, according to the invention, a suspending fluid can be added to a feedstock to produce a fermentation suspension. The suspending fluid can have sufficient liquid content to suspend the feedstock, and thereby reduces, and in some embodiments, largely eliminates the need for fresh process water. In certain embodiments, the suspension fluid contains no more than 20%, 10%, 5%, 2%, 1%, or substantially no exogenously added fresh water and/or commercial nutrient supplements.

The suspending fluid may include solid materials therein, including organic material that has at least partially been anaerobically digested. These solid materials contain nitrogen, and can in some embodiments eliminate the need for nutrient supplementation.

The suspending fluid may also include one or more types of anaerobic microorganisms. In certain preferred embodiments, the suspending fluid is substantially free of non-anaerobic microorganisms, which can be advantageous because aerobic microorganisms can interfere with fermentation processes (e.g., by consuming the feedstock).

In some embodiments, the suspending fluid can be biodigestate produced by the anaerobic bio-digestion of organic waste. Organic waste can be, and generally is, a mixture of discarded organic material having relatively low commercial value. Organic waste can include by-products from various industries, including agriculture, food processing, animal and plant processing, and livestock. Examples of organic waste include, but are not limited to: livestock manure, animal carcasses and offal, plant material, wastewater, sewage, food processing, and any combination thereof. Organic waste can also include human-derived waste, such as sewage and wastewater, discarded food, plant, or animal matter, and the like.

In certain embodiments, the suspending fluid may be fractioned from an anaerobic biodigestate, such that selected fractions are used in the subject methods.

For example, in certain embodiments, the fractioned anaerobic biodigestate is a liquid fraction generated by removing substantially all solids (e.g., greater than 91%, 93%, 95%, 97%, 99%, or close to 100%) from the anaerobic biodigestate. This can be done by, for example, passing the anaerobic biodigestate through a FAN screw press, or other equivalent mechanical devices. The liquid fraction resulting from this process may be used directly in the instant invention.

In certain embodiments, the liquid fraction contains about 1, 2, 3, 4, 5, 6, 7, 8, 9, r 10% (e.g., 3-9%) solids.

In certain embodiments, such liquid fraction may also be further fortified by a nutrient recovered from the anaerobic biodigestate. Such nutrients, including nitrogen or phosphate nutrients, may be obtained (e.g., isolated, purified or enriched) from the liquid fraction of the anaerobic digestate using methods known in the art.

In other embodiments, the fractioned anaerobic biodigestate may be an ultrafiltration concentrate (UFC) or an ultrafiltration permeate (UFP) generated from a liquid fraction of the anaerobic biodigestate, wherein the liquid fraction is generated by removing at least part of, or substantially all solids from the anaerobic biodigestate.

An anaerobic bio-digester can be used to convert or extract useful products from organic waste. Anaerobic bio-digesters can include an enclosed container, which can be a vat or vessel or housing, where anaerobic bio-digestion of organic waste takes place. The anaerobic bio-digester is enclosed generally to prevent exposure to air, or other atmospheric or local contaminants. Many anaerobic bio-digestion facilities and systems are known (e.g., horizontal or plug-flow, multiple-tank, vertical tank, complete mix, and covered lagoon digesters) and any of these can be suitable for purposes of the present invention.

In certain embodiments, the anaerobic bio-digester is the integrated system described in the co-pending U.S. Ser. No. 12/004,927, filed on Dec. 21, 2007, entitled “INTEGRATED BIO-DIGESTION FACILITY.” The entire content of the co-pending '927 application is incorporated herein by reference.

The anaerobic bio-digestion of organic waste can be performed by anaerobic organisms, which can, as described hereinabove, thereby produce biogas and biodigestate (also known as anaerobic digestion effluent). Biogas generally contains a mixture of gaseous methane, carbon dioxide, and nitrogen (which can be in the form of ammonia), but may also contain quantities of hydrogen, sulfides, siloxanes, oxygen, and airborne particulates, and is itself a useful product that can be combusted to produce energy.

In addition to biogas, biodigestate can be produced as a result of the anaerobic bio digestion of organic material. Biodigestate can be a mixture of a variety of materials, and can include organic material not digested by the anaerobic organisms, by-products of anaerobic bio digestion released by the organisms, and the organisms themselves. For example, the biodigestate can include carbohydrates, nutrients (such as nitrogen compounds and phosphates), other organics, wild yeasts, and large amounts of wastewater. In some embodiments, the solid content can be about 5-9% by weight, or about 5-6% by weight. The biodigestate is sufficiently digested so that it is substantially free of non-anaerobic organisms, which may be eliminated by consumption by the anaerobic organisms, the conditions of the anaerobic bio-digestion (which in addition to the substantial absence of oxygen, can include a predetermined temperature and pH set based upon the optimal living conditions of the anaerobic organisms), or a combination thereof.

The amount of each component within the biodigestate can, in some embodiments, be adjusted. For example, the amount of time the organisms are exposed to the organic material can be varied to alter the amounts of undigested organic material and anaerobic bio-digestion by products.

In some embodiments, the biodigestate can be transported without being stored to the ethanol feedstock for suspension. This can be done, for example, by using a pipe. These embodiments can be advantageous because they can reduce the risk of contamination of the biodigestate by non-anaerobic organisms.

As stated hereinabove, the fermentation suspension may already contain anaerobic organisms. Alternatively, anaerobic microorganisms suitable for ethanol production may be inoculated to the culture.

The fermentation suspension may additionally contain other microorganisms that can interfere with fermentation by, for example, digesting the feedstock and/or digesting the organisms performing the fermentation. These organisms can, however, be sensitive to pH. Thus in certain embodiments, the pH of the fermentation suspension can be adjusted such that the growth of the interfering microorganisms are substantially suppressed. This suppression entails preventing such interfering microorganisms from disrupting/inhibiting with fermentation of the feedstock into ethanol. In some embodiments, this suppression can be performed by killing the interfering microorganisms. In some embodiments, the pH can be adjusted to below 6.0. In certain preferred embodiments, the pH can be adjusted to fall in the range of 4.0 to 5.0.

The fermentation suspension can be fermented to produce ethanol under conditions (pH, temperature, etc.) conductive for ethanol production. The methods of the invention can be advantageous because the suspending fluid used reduces or eliminates the need for fresh process water, nutrient supplementation, or both. The subject method can also be advantageous because ethanol production can be increased due to the presence of fermentable material within the suspending fluid (but is lacking in fresh water).

In certain embodiments, the post fermentation beer may be distilled directly to collect ethanol without pre-removal of solids from the beer. This further reduces the cost of operating the ethanol plant according to the instant invention.

Wet Distillers Grains (WDG) are the remaining portions of the feedstock wheat that was added to the ethanol process after the distillation is complete. Most of the starch from the wheat is converted to ethanol by the microorganism, while the proteins and any lipids remain unused. These remaining portions of the grain are valuable and palatable as feed for cattle.

Therefore, in certain embodiments, the method of the invention contemplates building an integrated ethanol plant at the vicinity of an animal feedlot, wherein there is no need to use large amounts of energy to dry the wet distillers grains for long shelf life the way many ethanol plants are forced to. In addition, there will be no need to use large quantities of fuel to transport the distillers grains long distances to far away markets or feedlots. Instead, distillers grains can be sent to the nearby feedlot and consumed wet by the farm animals such as cattle. This configuration/combination not only provides major energy savings to the ethanol plant, but also reduces the amount of fresh drinking water the cattle consume.

In certain embodiments, the suspension fluid is added to the feedstock in multiple step, e.g., two steps. For example, in the first step, about 75% of the suspension fluid is added to the feedstock, e.g., high-starch wheat, before the liquidation step using alpha-amylase. The remaining 25% may be added post-liquidation, but before saccharification using glucoamylase.

The amount of the feedstock used may also be optimized. In certain preferred embodiments, the amount of the high-starch wheat is added up to about 28% (w/v) in the suspension fluid.

Systems designed for carrying out the methods of the invention may include an anaerobic bio-digester, wherein organic waste material produced therefrom can be subject to anaerobic biodigestion to produce biodigestate and biogas, as noted hereinabove.

As noted hereinabove, feedstock can contain complex sugars, such as polysaccharides, cellulose, or hemicelluloses, that generally can be hydrolyzed by specific chemical reagents to produce more easily fermentable sugars. In certain embodiments, at least a portion of the biodigestate can be transported as biodigestate to a hydrolysis unit, wherein it can be mixed with feedstock to produce feedstock suspension. Because the biodigestate contains material, such as cellulose or hemicelluloses, for example, that can be hydrolyzed, more sugar can be produced in hydrolysis than if fresh water is used to create the feedstock suspension. In some embodiments, the hydrolysis can be done by using one or more enzymes, such as alpha-amylase, glucoamylase, cellulase, xylanase, and/or acid proteolytic enzyme. In some embodiments, the hydrolysis can also be done using acid. In some embodiments, the hydrolysis can be done using heat, in the form of steam. Hydrolyzed feedstock suspension can be the result, which contains a more simple sugars that can be fermented to produce ethanol.

In certain embodiments, the suspending fluid is substantially free of exogenously added fresh water or nutrient supplements.

At least a portion of the biodigestate can be transported to a fermentor. Within the fermentor, biodigestate or fractions thereof can be mixed with feedstock, and ethanol can be produced after fermentation.

The invention also provides an exemplary process for hydrolyzing a feedstock in accordance with an embodiment of the present invention.

For example, a suspending fluid including organic material that has at least partially been anaerobically digested, and preferably containing one or more anaerobic microorganisms suitable for ethanol production, and which is substantially free of non-anaerobic microorganisms, can be added to a feedstock (such as corn or wheat, preferably high-starch wheat) to produce a feedstock suspension.

As described hereinabove, the feedstock can be hydrolyzed. In the embodiment described above, without being limited to specific order or repetition of steps, one or more steps of mechanical grinding or milling of the feedstock may be performed, one or more enzymes may be added, and the feedstock may be heated (preferably by steam). All these steps can be performed in the subject suspending fluid, preferably without any exogenously added fresh water and/or nutrient supplements. The feedstock suspension is hydrolyzed such that at least a portion of the polysaccharides therein are converted into simple sugars, which can subsequently be fermented to produce ethanol. While not wishing to be bound by any particular theory, the suspension fluid contains certain complex polysaccharides, such as cellulose or hemicelluloses that can be digested by the added enzymes to produce simple sugars.

While certain preferred illustrative embodiments of the invention are described above, it will be apparent to one skilled in the art that various changes and modifications may be made without departing from the invention. The appended claims are intended to cover all such changes and modifications that fall within the true spirit and scope of the invention.

### Examples

Having generally described the invention, Applicants refer to the following illustrative examples to help to understand certain aspects of the generally described invention. These specific examples are included merely to illustrate certain aspects and embodiments of the present invention, and they are not intended to limit the invention in any respect. Certain general principles described in the examples, however, may be generally applicable to other aspects or embodiments of the invention.

Examples described herein below demonstrate that integration of bioethanol facilities with feedlots and the IMUS (Integrated Manure Utilization System) technology is an excellent way of sharing infrastructure and using by-products on site. This integration increases the value of manure in the form of power and heat, which value is magnified through ethanol plant usage. The value also translates into significant reductions in the utility costs of the ethanol plant, and helps to make small ethanol plants co-exist with large feedlots in a balanced feed/by-product relationship.

The study was at least partly based on the analysis of the following integrations:
Ethanol production to feedlot operation: distiller wet grain and thin stillage
Ethanol production to IMUS process: low grade heat (<50° C.) and thin stillage
IMUS process to ethanol production: electricity and heat
IMUS process to ethanol production: digestate
IMUS process to feedlot operation: electricity
Feedlot operation to IMUS process: manure

Results from this study show that anaerobic digestate can be used to replace fresh water and fertilizer utilization for bioethanol production. Based on the data in this study, one can improve the economic viability of the bioenergy clustering model by, i.e., creating an eco-farm or bioindustrial network where all waste streams or related by-products are utilized. Ultimately, the system can be used to turn outputs into value-added products, e.g., beef, heat, bioethanol, bio-fertilizers, electricity, and collectable food-grade CO₂, in environmentally responsible ways.

### Example 1

### Anaerobic Digestate (AD) and Fractions Thereof Support Fermentation

The example shows that anaerobic biodigestates (AD) can replace fresh water for bioethanol production.

Four different separations of AD from IMUS™ demonstration plan in Vegreville (Alberta, Canada) were collected, including fresh anaerobic digestate (AD), FAN-separated digestate (FSD), and permeate (UFP) and concentrate (UFC) of FSD through ultra-filtration.

Specifically, FSD (FAN Separated Digestate) can be generated by using a screw press (such as a FAN brand screw press) or other similar mechanical devices to separate the digestate into two fractions—liquid fraction and solid faction. The liquid fraction is the FSD in this study. It contains about 5-7% total solids.

UFP/UFC can be generated by passing the FSD fraction through ultrafiltration. The permeate (UFP) is a relatively clean liquid (mostly water). The concentrated remains of whatever passed through the ultrafiltration system is designated UFC.

For small scale lab production, such as when used in this example, the UFP and UFC fractions were generated using a lab system that does not contain lime before the ultrafiltration system. In a typical run, a unit of the FAN-separated liquid digestate generated about 80% permeate and 20% concentrate.

Three pilot experiments were conducted to show:

(1) effect of AD on yeast fermentation of granulate sugar (food grade),

(2) ability of AD to ferment granulate sugar without yeast, and,

(3) ethanol yielding in comparison with tap water collected in laboratory.

Specifically, granulate sugar was dissolved in AD (pH˜8.1) and tap water (pH˜5.5) to a concentration of about 28 g/dl, respectively, and pH was adjusted to ˜5.4 with 12 N HCl. Fermentation was conducted in 1.0 liter volume in a 3.5 liter fermenting bottle for 14 to 24 days. Fermenting process was observed daily by measuring change in specific gravity of the mixtures using a hydrometer. The potential ethanol content (% volume) was calculated using Oechsle Scale (see, for example, en.wikipedia dot org/wiki/Oechsle_scale).

The Oechsle Scale is a hydrometer scale measuring the density of grape must, which is an indication of grape ripeness and sugar content used in wine-making. It is named for Ferdinand Oechsle and it is widely used in the German, Swiss and Luxemburgish wine-making industries. On the Oechsle scale, one degree Oechsle (° Oe) corresponds to one gram of the difference between the mass of one liter of must at 20° C. and 1,000 gram (the mass of 1 liter of water). For example, must with a mass of 1084 grams per liter has 84° Oe. The mass difference between equivalent volumes of must and water is almost entirely due to the dissolved sugar in the must. Since the alcohol in wine is produced by fermentation of the sugar, the Oechsle scale is used to predict the maximal possible alcohol content of the finished wine.

Selected samples were sent to a quality control (QC) lab in Alberta Centre for Toxicology (ACFT, University of Calgary) for ethanol analysis using a gas chromatograph (GC, HP6890) and a Flame Ionization Detector (FID).

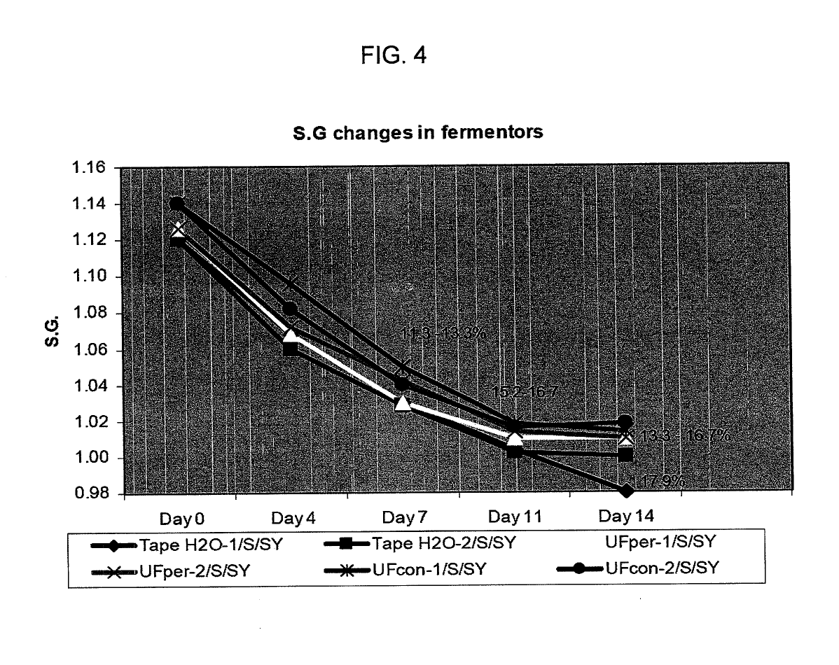

Results showed that, in comparison with tap water, there was no significant inhibitory effect of AD on yeast-driven fermentation for ethanol production. Potential ethanol yielding was around 13 to 16.7% in different ADs and ˜18% in water control (FIG. 4). Different ethanol contents were detected when different separations of AD with the same concentration of sugar were fermented, the highest seen in UPC (13.7 g/dL) and lowest in UFP (10.2 g/dL) during a 24-day fermentation (Table 1).

As a negative control, there was almost no ethanol produced under fermentation condition up to 24 days, when water and sugar were mixed without adding yeast (0.3 g/dL). However, 8.0 g/dL of ethanol was produced in the mixture of UFC and sugar without yeast, indicating that some components in UFC could facilitate fermentation. In addition, in UFP/sugar mixture without yeast, ethanol content was much lower (1.5 g/dL). This result suggests that some anaerobic microbes in the UFC/sugar mixture without yeasts assisted fermentation during the process.

A single step distilling experiment also showed that UFP and UFC beer could be distilled to yield clear ethanol with concentration of 70-71 g/dL (Table 1).

TABLE 1
Concentration of ethanol determined by GC and FID from
different fermentation groups up to 24 days at 22° C.

| | | | Ethanol | | S.G | BP | Ethanol |
|---|---|---|---|---|---|---|---|
| | | Ethanol | (g/g | Ethanol | in 1^{st} | @ | (g/dl) |
| ID | Content | (g/dL) | glucose) | (%) | Distill | ° C. | DS |
| 1 | H₂O + S | 0.34 | — | 0 | | | |
| 2 | UFcon + S + | 13.69 | 0.022 | 15.3 | | | |
| | FY | | | | | | |
| 3 | UFcon + FY | 0 | — | 0 | | | |
| 4 | UFper + S + | 10.22 | 0.019 | 13.4 | 0.8 | 76-78 | 70 |
| | FY | | | | | | |
| 5 | UFper + FY | 0.65 | — | 0 | | | |
| 6 | UFcon + S | 8.0 | 0.014 | 13.9 | 0.83 | 76-78 | 71 |
| 7 | UFper + S | 1.5 | 0.003 | 0 | 0.98 | 94-98 | |

Legends: Fy: Yeast; BP: boiling point; DS: distilled; ethanol (g/dL) measured by GC, ethanol (%) measured by hydrometer.

In conclusion, this example demonstrated that: (1) anaerobic digestate can be utilized as water replacement for bioethanol fermentation; (2) as total solids increased (UFC>UFP) in AD, ethanol concentration increased as well; and 3) post fermenting beer from AD was distillable to produce clear ethanol without pre-removal of solids from the mixture.

### Example 2

### Wheat Conversion in AD and Tape-water

This example demonstrates that AD does not inhibit alpha-amylases and glucoamylase during the conversion process from wheat to glucose. It also provides a comparison between the conversion rates of tape-water and AD when they were used as media.

Conversion from wheat or other crops to starch and then to glucose is the critical step for bioethanol production, since the amount of glucose will be directly related to the content of ethanol in the beer. Typically, an average conversion rate from wheat to glucose in bioethanol industry is around 56%.

Two most important enzymes during the conversion process are alpha-amylase and glucoamylase. The former catalyzes wheat to starch, the latter catalyzes starch to glucose. Two commercial converting enzymes, alpha-amylase (Spezyme XTRA) and glucoamylase (G-ZYME™ 480 ethanol) from Genencor® Inc., were used in two-step conversion experiments. D-glucose assay was adapted for evaluating the conversion rate of wheat in AD and water.

Specifically, wheat (soft white wheat-Andrew) ground using a hammer mill was obtained from Highmark Renewables Research. Different contents of unscreened wheat were prepared both in AD and tap water. Final concentrations for different treatment groups were 70, 140, 175, and 280 grams of wheat/1 liter of medium. Twelve experiments were set up in 1.0 liter medium using 2.0 liter beakers.

The first step of liquefaction by Spezyme XTRA was carried out at 85° C., pH 5.0 to 6.0 for 60 minutes, and the second step of saccharification by G-ZYME™ 480 was at 60° C., pH 4.0 to 4.5 for 30 minutes, respectively, after dose and reaction time were optimized. Samples were taken before and after two enzymes were added, and were centrifuged at 4,750 rpm for 15 minutes. The supernatant was collected and diluted with H₂O. The glucose concentration in the supernatant was determined by glucose assay, either by glucose assay kit (Sigma GAHK20-1KT) or YSI instrument with specific standard. Total carbohydrate in AD was also analyzed to determine whether there was available carbohydrate as substrate contributing to conversion.

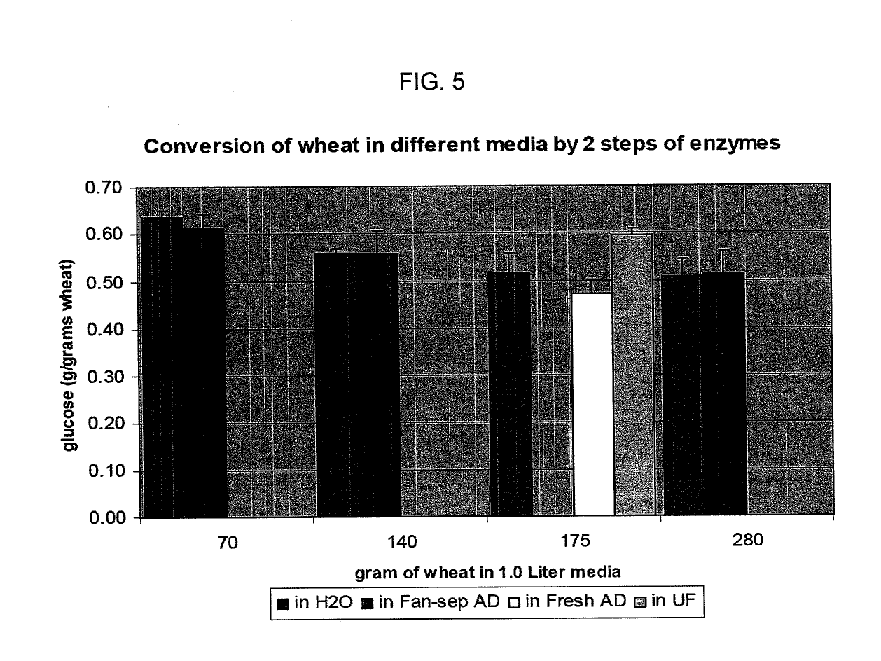

Results showed that there was no significant difference of glucose yield during wheat conversion by two enzymes in AD and tap water (FIG. 5). Efficiency of wheat conversion reached an average wheat conversion rate (˜56%). When different alpha-amylase and glucoamylase from different manufactures (Novozyme Inc) were tested, there seemed to be no discernible difference in conversion efficiency between Genencor and Novozyme enzymes in terms of glucose yielding (data not shown).

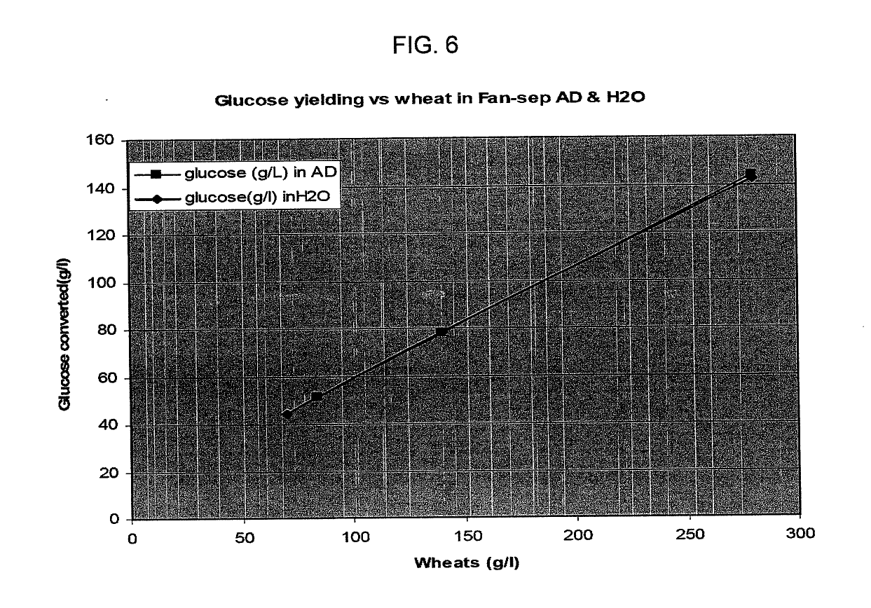

As concentration of wheat increased in the mixture (up to 28 g/dL in these experiments), glucose yielding increased correspondingly no matter wheat was converted in water or AD (FIG. 6). Content of total carbohydrate in AD was 4.11 g/dL in FSD. The supernatant of FSD contained only 0.12 g/dL (2.9% of original) of total carbohydrate after centrifugation.

In conclusion, no inhibitory effect of AD on two converting enzymes was observed during the wheat to glucose conversion process, as long as pH was controlled in an optimal range required for the reactions. Dose-dependent increase of glucose content was achieved as the amount of wheat was increased up to 28 g/dL in both AD- and water-wheat mixtures. Enzyme conversion efficiency was higher in low concentration wheat-medium mixture, but the difference was not significant.

As expected, small amounts of total carbohydrate existed in AD, but was not accessible for breaking down by the conversion enzymes. The carbohydrate is most likely in a non-dissolved form, and is assumed to be cellulose or hemi-cellulose (rather than starch-based polysaccharides).

### Example 3

### Ethanol Yields from Simultaneous Saccharification and Fermentation (SSF) Using AD and Tape-Water

Simultaneous saccharification and fermentation (SSF) studies were conducted to evaluate wheat-based bioethanol production in AD versus water. Since there were no negative impact of AD on glucose conversion from wheat and direct yeast-fermentation of sugar, ethanol yield in post-fermenting beer represents AD's effect on fermentation process.

The example provided direct comparison between final ethanol content of the beer from SSF using AD- and water-wheat mixtures. It also optimized the process of SSF in lab scale, and investigated which component in AD, nutrients, carbohydrate, proteases or microbes, contributed to ethanol production increase.

The SSF experiment was set up in 250 ml flasks containing 28 or 36 grams of dry wheat in 100 or 130 ml AD (FSD and UFP) and water, respectively. β3-glucanase/xylanase mixture (OPTIMASH™ BG from Genencor®, Rochester, N.Y.) was tested for catalysis of non-starch carbohydrates in wheat and/or AD in addition to two standard conversion enzymes used in Example 2. Liquefaction was processed at 85° C. for 1.0 hr as described above in Example 2. Then G-ZYME™ 480 (from Genencor®, Rochester, N.Y.) and BG were added at 60° C. for 30 minutes during saccharification. Super yeast X-press powder (AG grade for bioethanol) was pitched in distill water at 34° C. for 20 minutes, and then aliquots were added to the flasks with yeast nutrients to start ethanol fermentation.

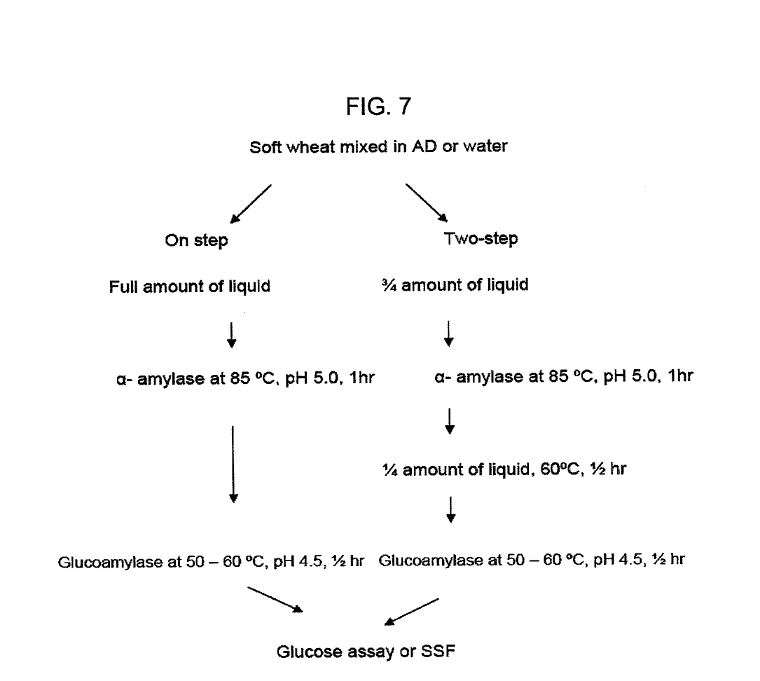

SSF Fermentation was set at 32° C. for 48 hours in water bath. Three SSF experiments were performed. The first experiment was aimed to test the effect of both AD and BG on final ethanol yield; the second was to test dose-dependent ethanol yield in 100 ml FSD with dry wheat of 12, 20 and 28 grams and BG, and the third was to test effect of two-step addition of AD or water (¾ of total volume of liquid for liquefaction and the ¼ total volume of liquid post liquefaction and before saccharification) on ethanol yield (FIG. 7).

Samples were sent to ACFT for ethanol analysis after centrifugation at 4,750 rpm for 15 minutes. 50 ml of post-fermentation mixture from each group were reserved for analysis of total solid (TS), volatile solid (VS), and total nitrogen (TKN) in the biowaste lab.

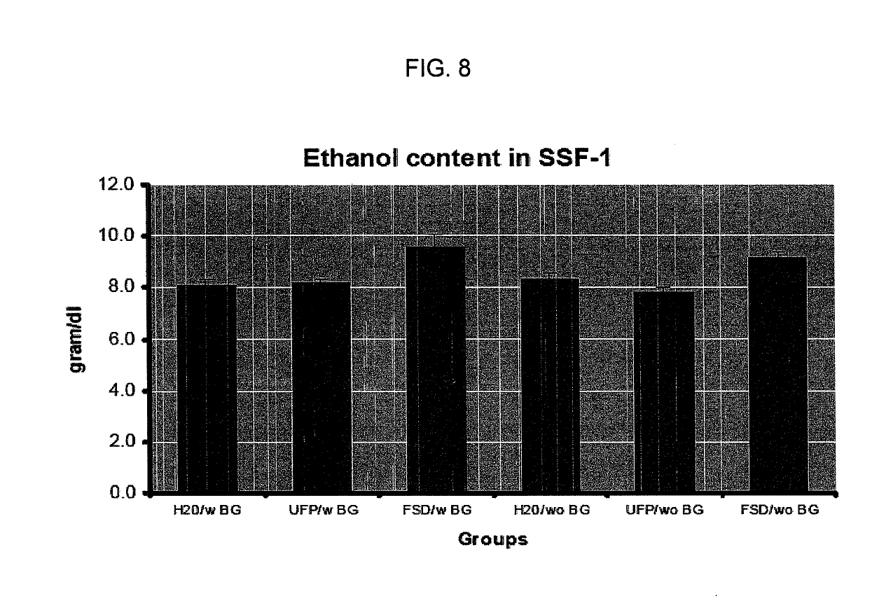

Somewhat surprisingly, in the SSF-1 experiment, the highest ethanol content was obtained in FSD with BG (9.57±0.5 g/dL) and without BG (9.20±0.17 g/dL), which was higher than that in water with and without BG (8.25±0.07 and 8.36±0.15 g/dl) (p<0.05 and <0.01, t-test), respectively. The ethanol content was 10 to 16% higher when FSD was used instead of water. There was no difference in ethanol yield between paired groups with and without BG (FIG. 8). Increase of ethanol content in AD-wheat fermentation seemed to have resulted from AD instead of β-glucanase/xylanase catalysis.

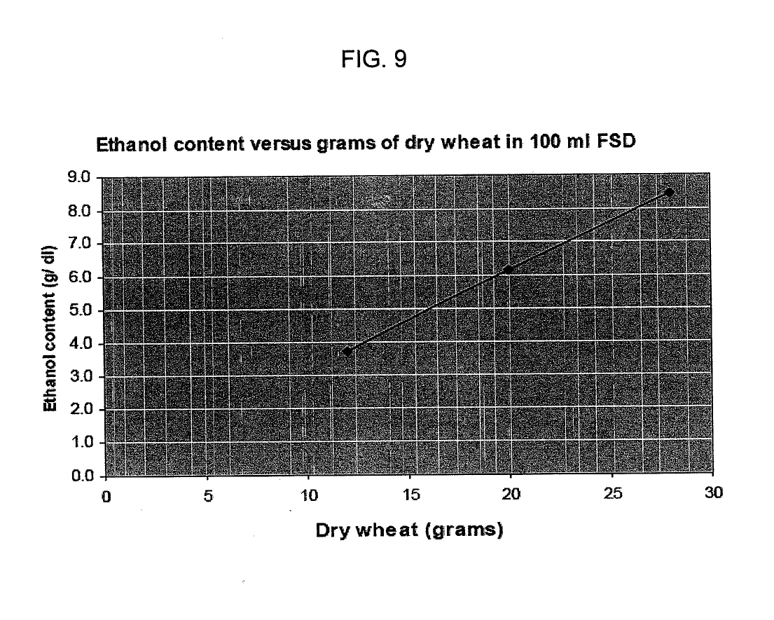

Dose-dependent increment of ethanol content was observed in the SSF-2 experiment. As dry wheat increased from 12 to 28 grams in 100 ml FSD, a good linearity of ethanol yielding was observed (FIG. 9). It was estimated that 0.3 grams of extra ethanol was produced per additional gram of dry wheat within this range.

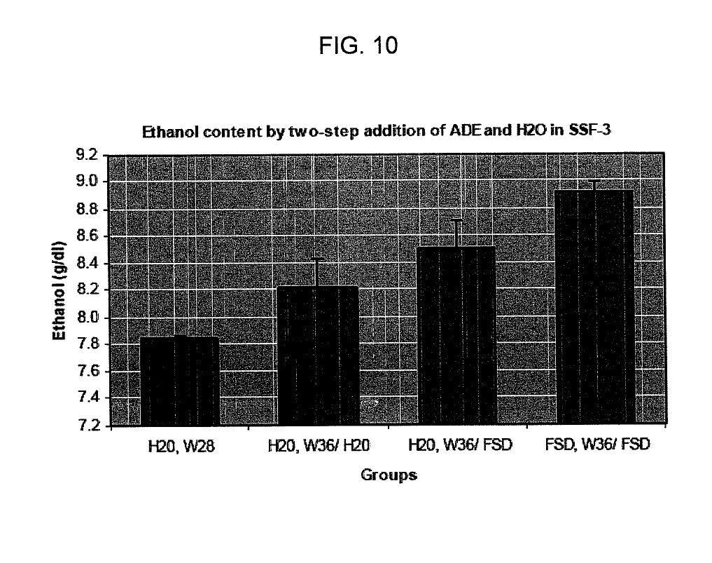

In the SSF-3 experiment, ethanol yield in a two-step procedure of AD or H₂O addition was compared with that of a one-step procedure. Interestingly, ethanol yield increased in all two-step procedures compared to the one-step procedures no matter FSD or water was added after liquefaction stage. With a similar final concentration of wheat (28 grams/dL) in fermentation mixtures, the highest ethanol content was seen in FSD/FSD mixture (8.93±0.07), secondly in H₂O (8.50±0.21), and then in H₂O/H₂O (8.21±0.22 g/dL). Ethanol content in wheat-H₂O mixture control reached only (˜7.9 g/dL) by the one-step procedure (FIG. 10).

Comparing the FSD/FSD mixture and the H₂O/H₂O mixture in the two-step procedure, ethanol content increased by 0.72 g/dL (Table 2). The results indicated that different procedures in conversion seemed to affect final ethanol yield.

TABLE 2
Statistical analysis (p value) on ethanol yield
in different groups (significant level p < 0.05)

| | H₂O | H₂O | H₂O | FSD |
|---|---|---|---|---|
| P value (n = 4) | W28 control | W36/H₂O | W36/FSD | W36/FSD |
| H₂O W28 control | | 0.045* | 0.008* | 0.0001* |
| (1-step) | | | | |
| H₂O W36/H₂O | | | 0.11 | 0.0008* |
| (2-step) | | | | |
| H₂O W36/FSD | | | | 0.08 |
| (2-step) | | | | |
| FSD W36/FSD | | | | |
| (2-step) | | | | |

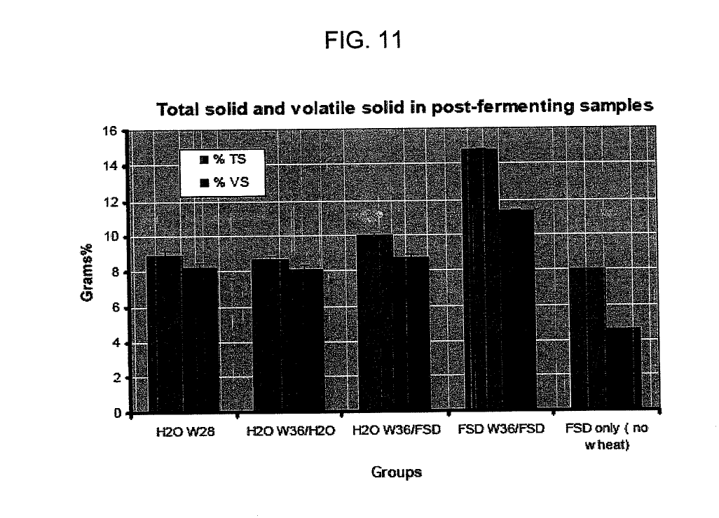

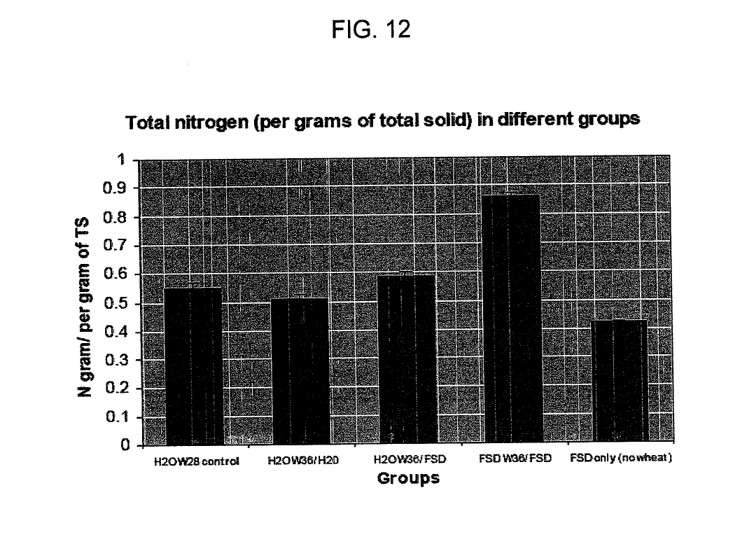

Total solid (TS) and volatile solid (VS) in post-fermenting samples were summarized in FIG. 11. With the same amount of wheat in the fermenting mixture, TS, VS (as % TS) were 14.8%, 76.76% in FSD/FSD and 8.69%, 92.86% in H₂O/H₂o group, respectively. Total nitrogen content in the post-fermenting solid was 0.87±0.007 grams/per gram of TS in FSD/FSD, and 0.51±0.016 grams/per gram of TS in H₂O/H₂O group (FIG. 12).

Having considered difference of total solid between the mixtures of wheat/FSD and wheat/H₂O, total nitrogen in the post-fermenting solid was much higher in the wheat/FSD than in the wheat/H₂O group, indicating that fermenting process was healthy and enhanced by using AD.

In conclusion, using FSD-wheat mixture, a single step of SSF could increase ethanol content 10-16% in post-fermenting sample. β-glucanase/xylanase enzyme mixture did not make a significant contribution for final ethanol yielding, indicating that limited amounts of non-starch carbohydrate substrate specific for the enzyme mixture were available in AD effluent. Two-step procedure of AD or H₂O addition led to an increase of ethanol yield compared to using the one-step procedure during SSF, especially in FSD/FSD group. It implies that: (1) wheat content in the mixture could be further increased over 28 grams/dL during liquefaction step; and (2) some microbes, biological molecules (such as proteolytic enzymes) and nutrients in raw AD do play a role for assisting yeast fermentation.

### Example 4

### Enhancement of Ethanol Yield Using Combination of Enzymes

It was observed that there were small amounts of carbohydrates in AD, but these carbohydrates could not be catalyzed by amylase, glucoamylase, and glucanase/xylanase. The example demonstrates that these carbohydrates in AD can be broken down by different enzyme combinations for enhanced bioethanol production. The example also provides an analysis regarding what such carbohydrates in AD are, and how much they contribute to ethanol production. The example further provides evidence to show that ethanol yield could be enhanced using protease during conversion and fermentation of AD- and H₂O-mixtures.

Two commercial cellulase mixtures from Genencor Inc., cellulase/xylanase (OPTIMASH™ XL), and ACCELLERASE 1000™, were tested in this experiment. In further experiments (results not shown), Novizyme's enzymes performed at least as well (if not better).

Assessment of conversion of non-starch carbohydrate in FSD was done by glucose assay (described in Example 2) and ethanol enhancement using SSF with modified procedure (as in Example 3). Conversion testing was set up in 250 ml flasks containing 100 ml of FSD or H₂O without wheat. Different doses of enzymes were added into liquids and incubated at appropriate temperatures and time following manufacture's instruction. α-amylase and glucoamylase were added then for liquefaction and saccharification. Glucose concentration was measured using YSI instrument. Experiment of SSF was performed in 250 ml flasks containing 28 grams of dry wheat (DW) in 100 ml FSD or water. OPTIMASH™ XL (0.01-0.1 ml per flask) and ACCELLERASE 1000™ (0.05-2.0 ml per flask) were added into the mixtures with α-amylase (Spezyme XTRA, 150 μl) and incubated at 50° C. for 24 hours before the saccharification step using G-ZYME™ 480 (100 μl). SSF Fermentation was carried out at 32° C. for 48 hours in water bath. To test the effect of protease (e.g., acid proteolytic enzyme, FERMGEN™) FERMGEN™ (20 and 100 μl per flask) was added after G-ZYME™ 480 and before adding yeast. Ethanol contents were measured by GC with FID in ACFT.

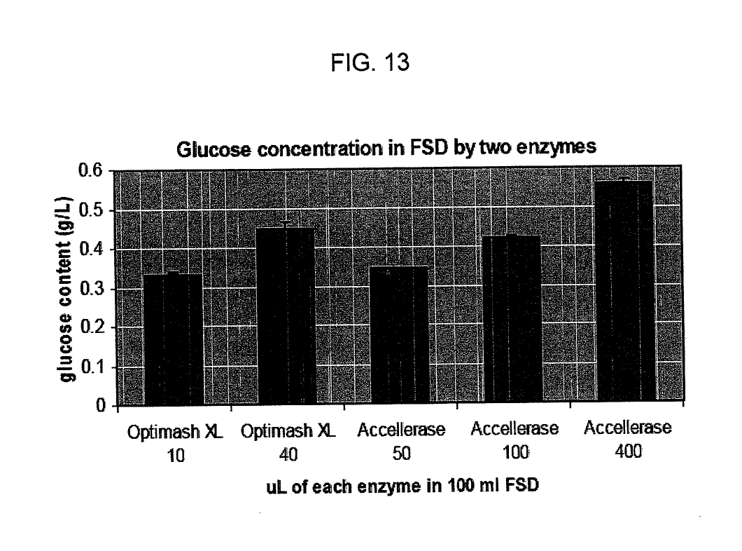

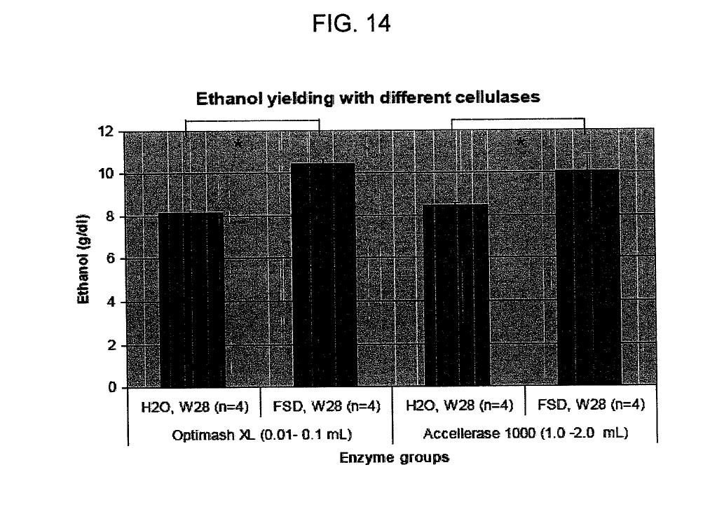

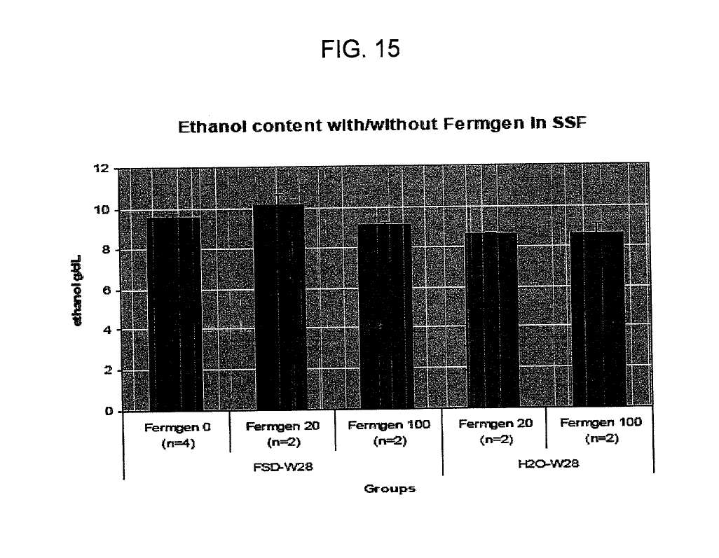

Results showed that dose-dependent increase of glucose content with two cellulase mixtures were observed in FSD but not in water without wheat. Highest yield of glucose was in ACCELLERASE 1000™ 400 μl (0.56 g/L) and then OPTIMASH™ XL 40 μl (0.45 g/L, FIG. 13). Almost no glucose in H₂O was detected after adding two enzymes (data not shown). Since two enzymes catalyze specifically the substrate of lignocellulosic biomass, increased glucose content indicated that there was lignocellulosic biomass in AD, although the amount was insignificant compared to the increased ethanol content after fermentation. When two enzymes were added into FSD- and H₂O-wheat mixture for SSF at 50° C. for a prolonged period of time (24 hrs), ethanol content was enhanced significantly in FSD with two enzymes (28% and 18% increase for OPTIMASH™ XL and ACCELLERASE 1000™, respectively) compared to that in H₂O with similar doses of enzymes (p<0.01, FIG. 14). Dose-dependent increase of ethanol yield was not seen between low and high doses, indicating that only limited amount of lignocellulosic biomass existed in FSD. Additional acid proteolytic enzyme (FERMGEN™) in the mixture enhanced ethanol content in post-fermenting beer slightly. The ethanol content increased 6% in FSD with 20 μL of FERMGEN™ per flask, compared to FSD without FERMGEN™. However, when compared to H₂O-wheat mixture with same dose of FERMGEN™, the ethanol content increase in FSD-wheat mixture was 17% (FIG. 15).

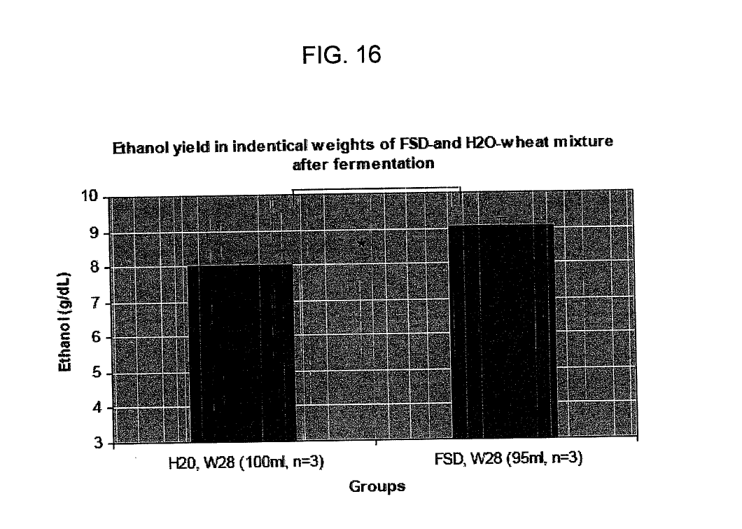

The FSD used in this experiment contained 5 to 7% total solid. Utilization of the same volume of FSD and water mixed with same amount of wheat will result in discrepancy of final volume of the beer after fermentation. In order to normalize the final ethanol yield, volume difference of the beer between two mixtures were analyzed. 5% less volume of beer was observed in FSD-wheat mixture than that in H₂O-wheat mixture. The volume correction factor was 0.95 for final ethanol yield when the same volume of FSD was used to replace water. Using FSD- and H₂O-mixture with the exact same weights, we found that the ethanol yield in FSD wheat mixture with a final volume of 95 ml increased ˜15% when compared to that of the H₂O-wheat mixture with a final volume 100 ml (FIG. 16).

In conclusion, ethanol yield was enhanced to about 28% or 18% by addition of cellulases, OPTIMASH™XL and ACCELLERASE 1000, respectively, via a modified liquefaction procedure at 50° C. for a long incubation time (24 hrs) of hydrolysis. These two enzymes catalyzed lignocellulosic biomass existed in AD, which contributed to final ethanol yield. An acid proteolytic enzyme assisted fermentation of FSD-wheat mixture to a lesser degree than that of H₂O mixture, indicating that some proteases already existed in FSD-wheat mixture and helped fermentation. These experiments provided further evidence that AD itself made major contributions to final ethanol yield through assisting enzymatic hydrolysis of wheat and improving fermentation by its microbes, proteases and nutrients. A volume correction factor 0.95 was used to normalize final ethanol yield when FSD was used as medium. Taken into consideration, final ethanol yield in FSD-wheat mixture from different fermentation experiments was 5 to 11% in Experiment 3 and 13 to 23% in Example 4.

In summary, results in these examples show that:

(1) anaerobic digestate (AD) has no inhibitory effect on a variety of converting/hydrolytic enzymes as well as the yeast-driven fermentation process;

(2) dose-dependent increase of glucose conversion was achieved as amount of wheat was increased up to about 28% (w/v), or even about 36% (w/v) in anaerobic digestate;

(3) ethanol content in post-fermenting beer increased as total solid increases in different separations of anaerobic digestate;

(4) simultaneous saccharification and fermentation (SSF) increased ethanol content by 5-11% in post-fermenting beer;

(5) ethanol content increased to 13-23% by addition of cellulase mixture and incubation at 30-50° C. (inclusive) for a prolonged catalytic time (24 hours);

(6) small amount of non-starch carbohydrate, such as lignocellulosic biomass, existed in anaerobic digestate;

(7) the two-step procedure for adding anaerobic digestate increased ethanol yield compared to the one-step procedure;

(8) post-fermenting beer was distillable to produce clear ethanol without pre-removal of solids;

(9) increased nitrogen content in post-fermenting solid could promote utilization of the stillage as fertilizer; and

(10) synergistic effect of microbes, proteases, and nitrogen in anaerobic digestate on fermentation plays a major role for ethanol enhancement.

### Example 5

### Animal Feed or Fertilizer Analysis

The “mash” or the wet distiller's grain-like material in the post-fermentation digestate and wheat can be used for animal feed (e.g., swine, poultry, fish, and cattle), optionally with fortified nutrient elements. The same material may also be used as fertilizer. This experiment shows that the “mash” has equivalent feed value compared to the usual wet distiller's grain (WDG) resulting from using fresh water alone. The experiment also shows that the mash has enhanced nutrient value as fertilizer compared to anaerobic digstate alone.

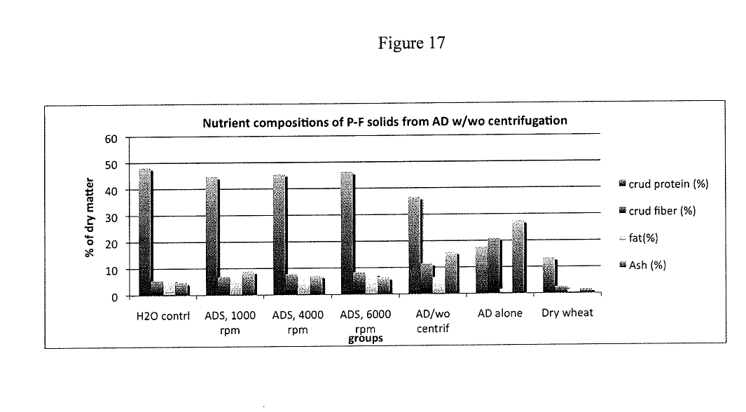

As shown in FIGS. 17, 18, and 19, “AD alone” represents the nutrient values of the anaerobic digestate alone before fermentation; “AD/wo centrif ” represents the nutrient values for whole AD fermented with wheat (“P-F” stands for “post fermentation”); “ADS nnn rpm” represents the nutrient values for centrifuged AD at different speeds and fermented with wheat and “H₂O control control” represents the nutrient values for wheat fermented in water.

In order to determine whether the resulting wet distiller's grain-like mash is also a nutritious animal feed, the protein, crude fiber, and fat contents of the mash is compared with WDG made from fresh water alone. FIG. 17 shows that the mash resulting from fermentation using centrifuged anaerobic digestate (ADS) has essentially the same quality as the WGD resulting for fermentation using fresh water. For example, crud protein was increased from 13% (in dry wheat) to 45-50% in fresh water control (H₂O control) and 43-47% (in ADS) post-fermentation. Total digestible nutrients, non-fibre carbohydrate and fat were compatible with the WGD from H₂O control. Furthermore, the following essential metal elements for animal feeding in post fermented solid with ADS were also equivalent or enhanced in comparison with post fermented solid in H₂O control, including calcium, magnesium and zinc. In addition, there is no mercury, lead or other unneeded elements in the post-fermentation solid. Therefore, the resulting stillage qualified as animal feed.

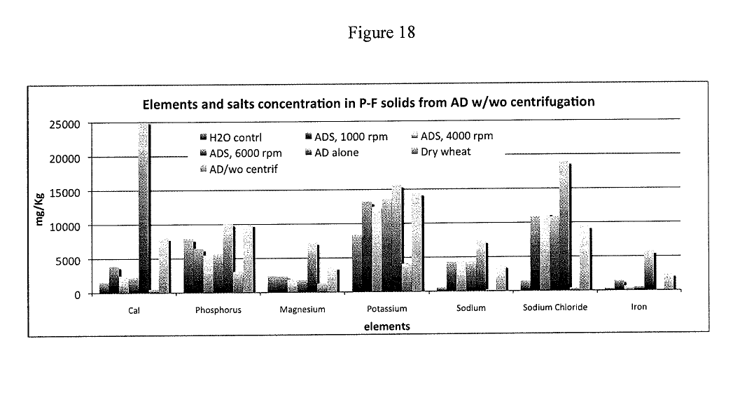

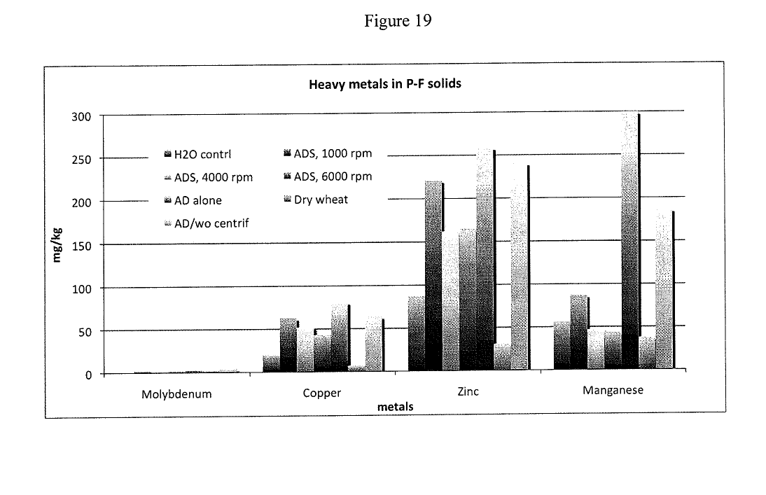

FIGS. 18 and 19 show the result of analyzing the various nutrient elements required in animal feeds as they are present in the various mash or WDGs. The results show that the various ADS batches contained slightly varied concentrations of the elements. Note that the concentrations of metal elements can be adjusted by using simple centrifugation at different speeds. The mash or WDGs with different contents of metal elements could directly feed animals during special growth phases to meet their physiological requirements.

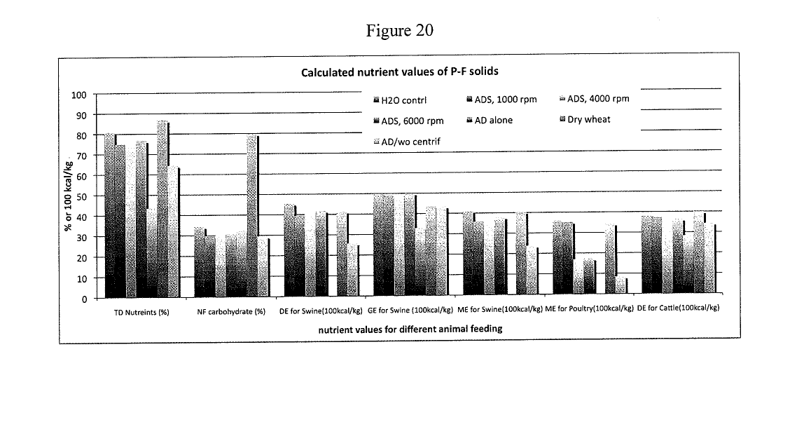

FIG. 20 shows the calculated animal feed values for the various ADS batches as compared to fresh water alone. The results show that the various ADS batches are at least as nutritious, if not more nutritious, than the water alone control.

It is apparent that, replacing fresh water with AD in ethanol fermentation not only fails to compromise the fermentation process, but expectedly result in wet distiller's grain-like mash that has enhanced nutritious as fertilizer compared to the digestate effluent without fermentation and mash or WDGs that resulting from using fresh water. Note that the nitrogen value is not shown in FIG. 17, but crud protein percentile per unit increased more than 60% compared to AD and dry wheat alone. All element contents were increased in post-fermented mash of AD with wheat in comparison with that in H₂O control fermentation. However, heavy metal element content was decreased in post-fermented mash of AD with wheat in comparison with AD alone without fermentation (FIG. 20). This will qualify the post-fermented mash or WDGs as the better fertilizer than the digestate effluent.

In addition, depending on the wheat concentration using in fermentation, the total volume of the mash usually increases about 30-50% compared to fresh water WDGs. Net mass yielding was significantly increased after fermentation as fertilizer. Meantime, the ash was reduced by 50% (from 30% to 15% as dry matter) in post-fermented mash of AD with wheat in comparison with AD alone (data not shown in the figures).

### REFERENCES

1. (S&T)² Consultants Inc. and Meyer Norris Penny LLP. Economic, financial, social analysis and public polices for bioethanol, Phase I report. Nov. 22, 2004.
2. Ethanol Short Course, North American Bioproducts Corporation, Feb. 11-15, 2008, Schaumburg, Ill.
3. Page I C. Anaerobic treatment of ethanol production wastes: 15 years of operating history and emerging applications. Fuel Ethanol Workshop, Jun. 26-28, 2007, St Louis.
4. Farrell A E, et al. Ethanol can contribute to energy and environmental goal. Science, 311: 506, 2006.
5. Hahn-Hägerdal B, et al. Towards industrial pentose-fermenting yeast strains. Applied Microbiology and Biotechnology, 74: 937, 2007.
6. Öhgren K. et al. Simultaneous saccharification and co-fermentation of glucose and xylose in steam-pretreated corn stover at high fiber content with saccharomyces cerevisiae TMB 3400. Journal Biotechnology 126: 488, 2006.
7. Sommer P et al. Potential for using thermophilic anaerobic bacteria for bioethanol production from hemicellulose. Biochemical Society Transactions (part 2), 32: 283, 2004.
8. Doxon L E, Money and Energy, in the Alcohol Fuel Handbook, p. 15 to 20, 2001, Infinity Publishing.com.
9. Hickey B and Motylewski M. Sustainable alternatives for whole stillage management. Fuel Ethanol Workshop, Jun. 26-28, 2007, St Louis.
10. Hirl P J. Self-generation of energy for ethanol production from distiller's grains anaerobic digestion. Fuel Ethanol Workshop, Jun. 26-28, 2007, St Louis.
11. Jenson E and X. Li. TECHNICAL FEASIBILITY STUDY OF COUPLING ETHANOL PRODUCTION WITH BIOGAS PRODUCTION/UTILIZATION. IRAP REPORT, March 2003.
12. Khan E and Yang P Y. Bioethanol production from dilute feedstock, Bioresource Technology, 47: 29, 1994.
13. en.wikipedia dot org/wiki/Oechsle_scale.

All references cited herein are incorporated by reference.

### ABBREVIATIONS IN THE REPORT

AD Anaerobic Digestate effluent
GHG Greenhouse Gas
SSF Simultaneous Saccharification and Fermentation
IMUS Integrated Manure Utilization System
FSD FAN-Separated anaerobic Digestate
UFP Ultra-Filtration Permeate
UFC Ultra-Filtration Concentrate
QC Quality Control
ACFT Alberta Centre For Toxicology
GC Gas Chromatography
FID Flame Ionization Detector
DW Dry Wheat
dL Deciliter
TS Total Solid
VS Volatile Solid
TKN Total Kjeldahl Nitrogen
gPM gallon Per Minute
HCl Hydrochloric acid
μL Microliter
mL Milliliter
L Liter

1. A method for producing ethanol, comprising:
(1) adding a suspending fluid to a feedstock to produce a fermentation suspension, wherein the suspending fluid comprises an organic material that has at least partially been anaerobically digested;
(2) adjusting the pH of the fermentation suspension, if necessary, to a value conductive for fermentation; and
(3) fermenting the fermentation suspension to produce ethanol, wherein the suspending fluid is substantially free of (exogenously added) fresh water or nutrient supplement.

2. The method of claim 1, further comprising inoculating the fermentation suspension with a microorganism capable of fermenting the fermentation suspension to produce ethanol.

3. The method of claim 2, wherein the microorganism is yeast.

4-6. (canceled)

7. The method of claim 1, wherein the suspending fluid comprises anaerobic biodigestate or a fractioned anaerobic biodigestate.

8. The method of claim 7, wherein the fractioned anaerobic biodigestate is a liquid fraction generated by removing substantially all solids from the anaerobic biodigestate.

9-10. (canceled)

11. The method of claim 8, wherein the liquid fraction is further fortified by a nutrient recovered from the anaerobic biodigestate.

12. The method of claim 7, wherein the fractioned anaerobic biodigestate is an ultrafiltration concentrate or an ultrafiltration permeate generated from a liquid fraction of the anaerobic biodigestate, wherein said liquid fraction is generated by removing substantially all solids from the anaerobic biodigestate.

13. The method of claim 1, wherein the pH of the fermentation suspension is adjusted to below 6.0, or between 4.0 and 5.0.

14. (canceled)

15. The method of claim 1, further comprising distilling the post fermentation beer to collect ethanol without pre-removal of solids from the beer.

16. The method of claim 1, wherein the feedstock is high-starch wheat, corn, or other high-starch crop.

17. The method of claim 16, wherein said high-starch wheat, corn, or other high-starch crop is converted in the suspending fluid at least partially into simple sugars.

18. The method of claim 16, wherein the conversion comprises (with no particular order and no limitation on repeats) mechanical grinding, heating with steam, reacting with an acid, liquefaction by using alpha-amylase, and/or saccharification by using glucoamylase.

19. (canceled)

20. The method of claim 17, wherein about 75% of the suspension fluid is added before liquefaction, and about 25% of the suspension fluid is added post liquefaction and before saccharification.

21. (canceled)

22. The method of claim 1, further comprising adding cellulase, xylanase, and/or acid proteolytic enzyme to the suspension fluid.

23. The method of claim 22, further comprising incubation the fermentation mixture at 50° C. for about 24-72 hours.

24. The method of claim 16, wherein the wet distillers grains resulting from ethanol distillation is fed to a livestock animal as feed or used as fertilizers.

25. The method of claim 1, wherein the suspending fluid is substantially free of non-anaerobic microorganisms.

26. (canceled)

27. The method of claim 1, wherein said nutrient supplement is a nitrogen supplement.

28. The method of claim 1, wherein ethanol yield is enhanced or increased compared to an otherwise identical process using fresh water instead of the suspending fluid.

29. A method for hydrolyzing a feedstock, wherein the feedstock comprises polysaccharides and wherein the hydrolyzed feedstock yields more ethanol when fermented than prior to hydrolysis, the method comprising:
(1) adding a suspending fluid to the feedstock to produce a feedstock suspension, wherein the suspending fluid comprises organic material that has at least partially been anaerobically digested; and
(2) hydrolyzing the feedstock suspension such that at least a portion of the polysaccharides are converted into simple sugars,
wherein the suspending fluid is substantially free of (e.g., exogenously added) fresh water or nutrient supplement.

30. (canceled)
